(19) United States
(12) Patent Application Publication
(10) Pub. No.: US 2014/0014117 A1
(43) Pub. Date: Jan. 16, 2014

(54) CORD MANAGEMENT DEVICE AND METHOD OF USE THEREOF

(71) Applicant: Robert WEINBERG, Spring Grove, IL (US)

(72) Inventor: Robert WEINBERG, Spring Grove, IL (US)

(73) Assignee: ALLEGIANCE CORPORATION, McGaw Park, IL (US)

(21) Appl. No.: 13/548,667

(22) Filed: Jul. 13, 2012

(51) Int. Cl. A61B 19/10

(52) U.S. Cl. 128/849

(57) ABSTRACT

A cord management device adapted for use with a surgical drape including a mounting portion adapted to be affixed to a surface of the surgical drape, a substantially rigid arm having a fixed end and a free end, wherein the arm is coupled to the mounting portion at the fixed end, and the free end is free from the mounting portion, and wherein the mounting portion and the arm define a cord receiving portion. The device may also include a second arm having a fixed end and a free end, wherein the second arm is coupled to the mounting portion at the fixed end of the second arm, and the free end of the second arm is free from contacting the mounting portion, and wherein the mounting portion and the second arm define a second cord receiving portion.

### BACKGROUND OF THE INVENTION

1. Field of the Invention

Aspects of the present invention relate to cord management and more particularly to cord management during surgery.

2. Background

When performing surgery, practitioners often use a variety of surgical tools and devices that include cords, tubes, wires, and the like. For example, surgeries often include fluid management tubing, electrosurgery cords, etc. The term “cord” as used herein means any of such tubes, cords, wires, and the like that are present during various surgical procedures. It is critical during surgery that a practitioner is able to control the placement and positioning of the cords so that the cords do not interfere with the surgery.

Currently, a method of cord management includes securing the cords to a surgical drape through hook and loop fasteners and/or fenestrated tabs. In particular, as shown in FIG. 1, in a first known method, a hook and loop fastener 10 is used to secure the cords 12 to the drape 14. The hook and loop faster 10 includes hook on a substrate material that mates with loops on a second substrate. The loop side is bonded directly on the drape 14. To secure tubes within the fastener 10, the user must first place the cords 12 in between the loop portions of the drape, and then place the hook substrate side overtop the cords 12 such that the hooks engage the loops. However, this operation requires two hands or another person to assist. Additionally, the hook and loop method requires significant effort to adjust the cords because the hook and loop mating must be broken, at which time the loop portion may shed and deposit particulate around the cord management device.

As shown in FIG. 2, a second known method of cord management includes using a fenestrated tab 20 having a plurality of through holes 22 and a junction portion 26 formed between the through holes 22. The tab 20 is secured to drape 24 on one end and is free on an opposing end. In operation, the user feeds a cord or tube (not shown) through the holes 22 in the tab 20, either singly or doubled over on itself, to secure the position. Alternatively, the user folds the tab over the cord or tube, then clamps the tab 20 to the drape to secure the position. However, these methods require two hands to operate and once the cord is secured, it is difficult to readjust the cord positioning without completely removing the cord from the tab 20.

There remains a need in the art for a cord management device capable of being operated with one hand and that allows for easy adjustment of cords.

### SUMMARY OF THE INVENTION

Aspects of the present invention provide, among other features, a surgical drape system, comprising a surgical drape, and a cord management device affixed the surgical drape, the cord management device comprising a mounting portion contacting a surface of the surgical drape, an arm having a fixed end and a free end, wherein the arm is coupled to the mounting portion at the fixed end, and the free end is free from contacting the mounting portion, and wherein the mounting portion and the arm define a cord receiving portion.

In another variation, the present invention provides a method of securing a cord to a surgical drape, the method comprising providing a cord management device affixed the surgical drape, the cord management device comprising a mounting portion contacting a surface of the surgical drape, an arm having a fixed end and a free end, wherein the arm is coupled to the mounting portion at the fixed end, and the free end is free from the mounting portion, and wherein the mounting portion and the arm define a cord receiving portion, expanding the cord access path by applying a bending force on the arm, passing a cord through the cord access path, placing the cord in the cord receiving portion after passing the cord through the cord access path, and releasing the bending force on the arm, thereby retaining the cord within the cord receiving portion.

In yet another variation, the present invention provides a cord management device comprising a mounting portion affixable to a surface, a first arm having a fixed end and a free end, wherein the first arm is coupled to the mounting portion at the fixed end of the first arm, and the free end of the first arm is free from the mounting portion, a second arm having a fixed end and a free end, wherein the second arm is coupled to the mounting portion at the fixed end of the second arm, and the free end of the second arm is free from the mounting portion, wherein the mounting portion and the first arm define a first cord receiving portion, and wherein the mounting portion and the second arm define a second cord receiving portion.

Additional advantages and novel features of various aspects of the present invention will be set forth in part in the description that follows, and in part will become more apparent to those skilled in the art upon examination of the following or upon learning by practice thereof.

### BRIEF DESCRIPTION OF THE FIGURES

In the drawings:

FIG. 1 shows a prior art cord management device;

FIG. 2 shows another prior art management device;

FIG. 3 shows a top perspective view of a cord management device in accordance with aspects of the present invention;

FIG. 4 shows a bottom perspective view of the cord management device of FIG. 3; and

FIG. 5 shows a top view of the cord management device of FIG. 3 with cords retained therein;

FIG. 6 shows a top view of a cord management device in accordance with another aspect of the present invention;

FIG. 7 shows a top view of a cord management device in accordance with another aspect of the present invention;

FIG. 8 shows a top view of a cord management device in accordance with another aspect of the present invention;

FIG. 9a shows a top view of a cord management device in accordance with another aspect of the present invention;

FIG. 9b shows a top view of the cord management device of FIG. 9a in an unlocked position;

FIG. 10a shows a top view of a cord management device in accordance with another aspect of the present invention;

FIG. 10b shows a top view of the cord management device of FIG. 10a in an unlocked position; and

FIG. 11 shows a top view of a cord management device in accordance with another aspect of the present invention.

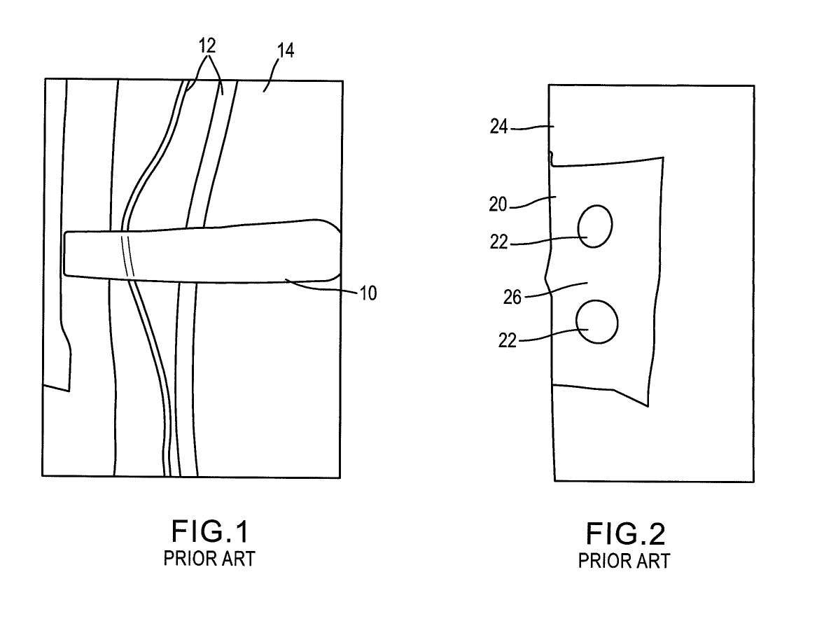

### DETAILED DESCRIPTION

Relative terms such as “lower” or “bottom” and “upper” or “top” may be used herein to describe one element's relationship to another element illustrated in the drawings. It will be understood that relative terms are intended to encompass different orientations in addition to the orientation depicted in the drawings. By way of example, if the apparatus depicted in the drawings is turned over, elements described as being on the “bottom” side of the other elements would then be oriented on the “top” side of the other elements. The term “bottom” can therefore encompass both an orientation of “bottom” and “top” depending on the particular orientation of the apparatus.

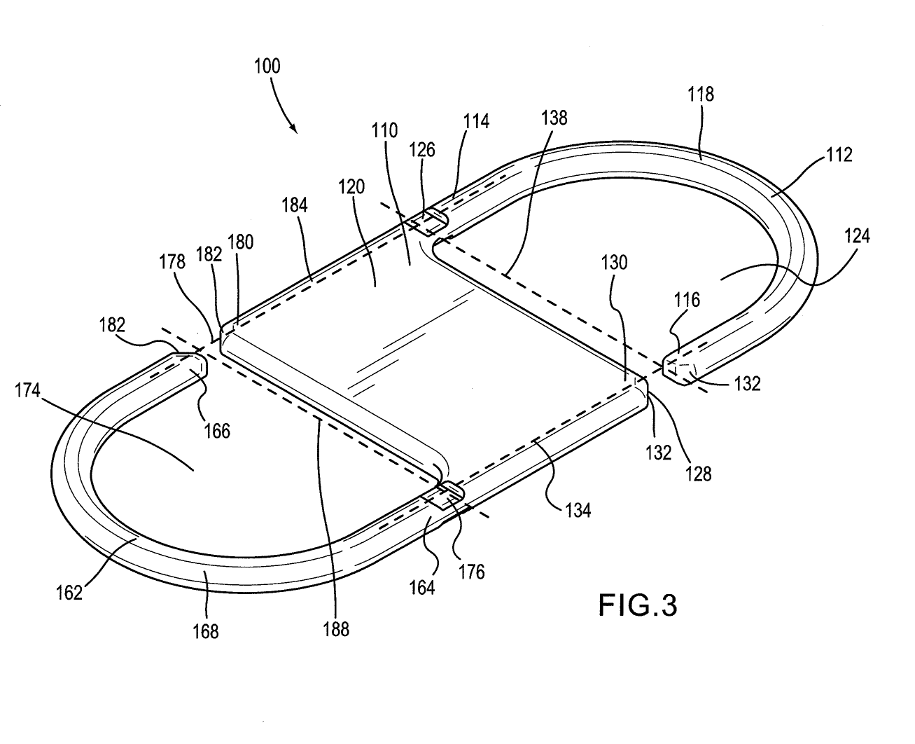

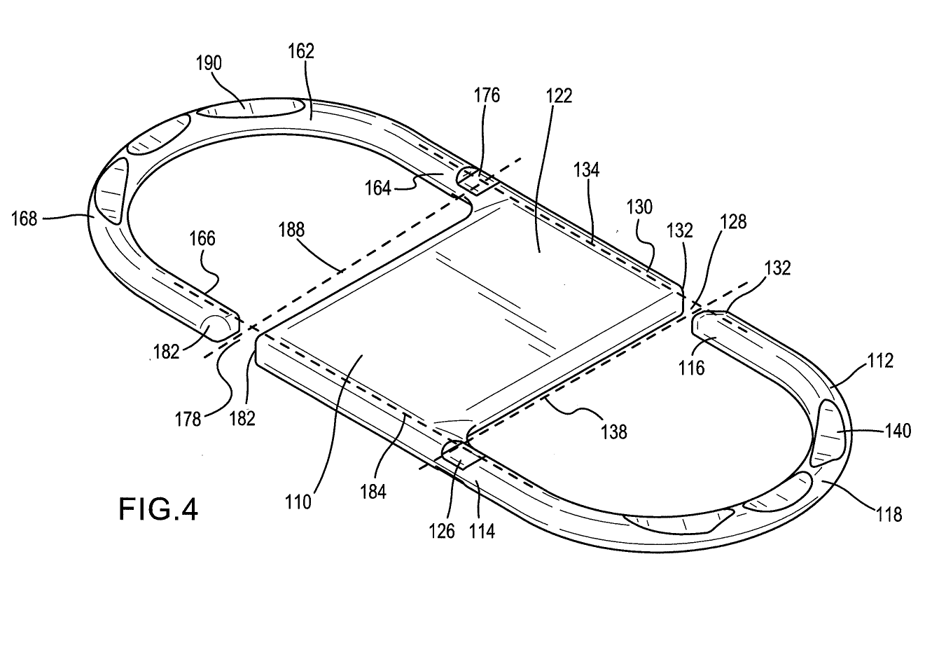

FIG. 3 shows a top perspective view, FIG. 4 shows a bottom perspective view, and FIG. 5. shows a top view of a cord management device 100 in accordance with aspects of the present invention. The cord management device 100 includes a mounting portion 110 mountable to a surface of a surgical drape 200 (FIG. 5), the mounting portion having a top surface 120 and a bottom surface 122. While the mounting portion 110 shown in FIGS. 3-5 comprises a substantially rectangular shape, any variety of geometries may be used. The bottom surface 122 of the mounting portion 110 may include a securing feature (not shown) to allow the mounting portion 110 to be mounted to the surgical drape 200. For example, the securing feature may include an adhesive covered by a removable film. In practice the film may be removed to expose the adhesive and then the bottom surface 122 may be placed on a desirable portion of the surgical drape 200. In another aspect, the securing feature may include hooks or loops that can be mated with corresponding hooks/loops on the surgical drape. In yet another aspect, the securing feature may include hooks that are sized to directly engage the fiber material of the drape 200. Alternatively, the mounting portion 110 may be mounted to the surgical drape 200 by the manufacturer by applying adhesive to the bottom surface 122 and then using a hot melt process to secure the cord management device 100 to the drape 200. Any suitable mounting method known in the art may also be used.

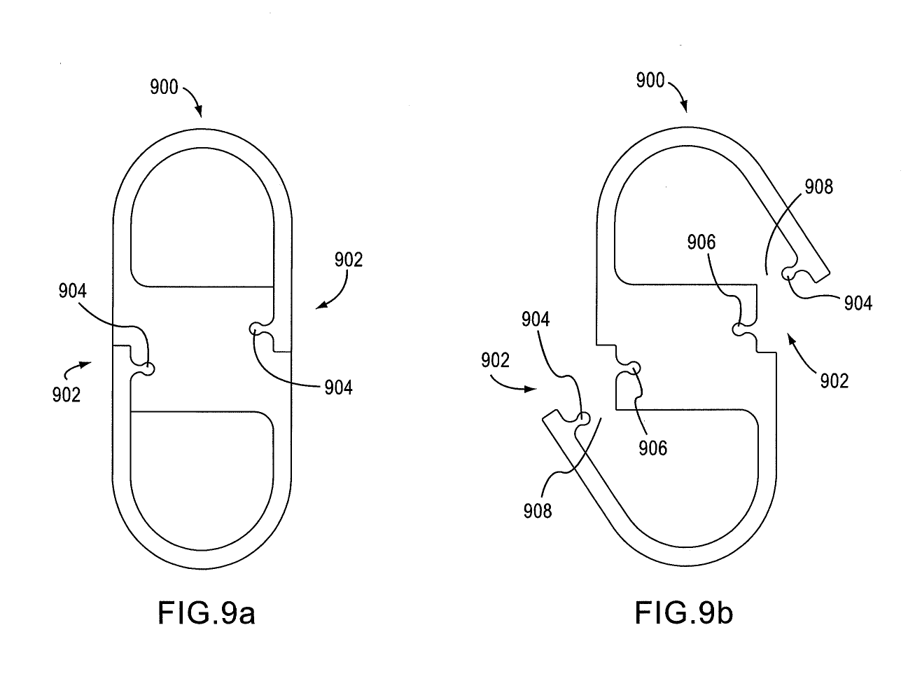

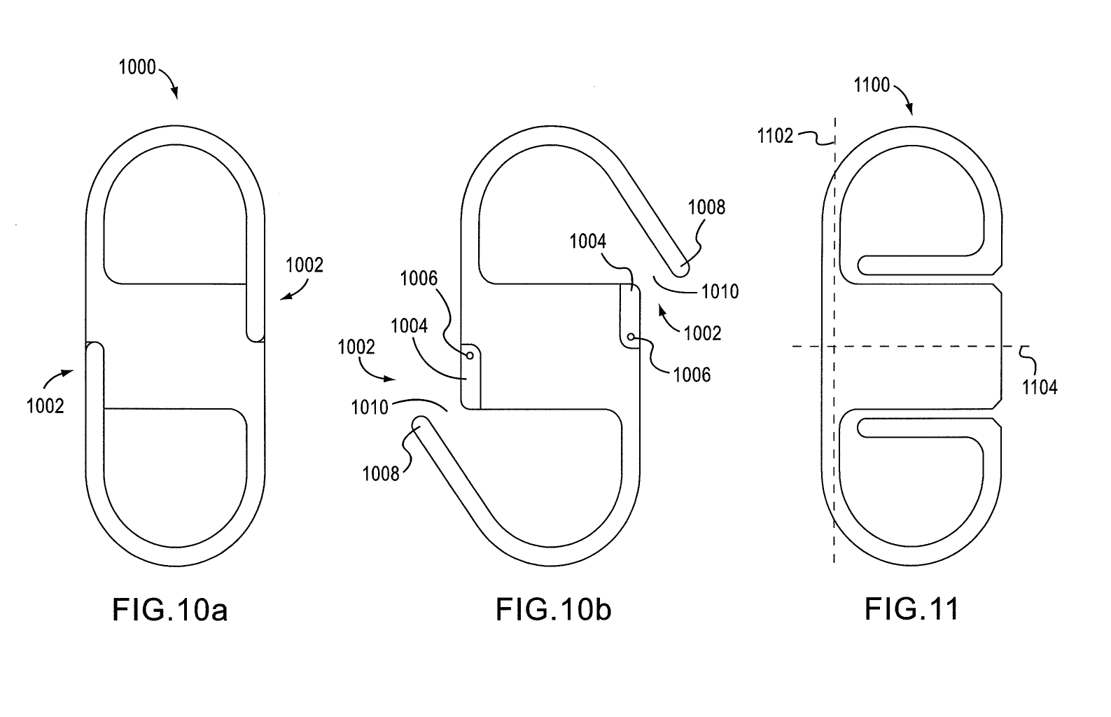

The cord management device 100 may further include an arm 112 having a fixed end 114 and a free end 116. The arm 112 may be coupled to the mounting portion 110 at the fixed end 114, while the free end 116 is free from contacting the mounting portion 110 or any other portion of the cord management device. The free end 116 may be free from contacting the mounting portion 110 or nay other portion of the cord management device as shown in the Figures. Alternatively, the free end may contact the mounting portion 110 or any other portion of the cord management device, so long as the free arm is not integrally formed with the mounting portion 110, but rather is either not fixed to the mounting portion 10, as is described in regard to various aspects of the present invention, for example, shown in FIGS. 3-8 and 11, or is detachably fixed to the mounting portion 10, as is described in various other aspects of the present invention, for example, shown in FIGS. 9a-10b. As shown in FIG. 3, the arm 112 may comprise a U shape, where the fixed end 114 and the free end 116 form the ends of the U. A curved portion 118 forms the curve of the U shape between the fixed end 114 and the free end 116. Other shapes, such as a square shape, may also be utilized. The free end 116 may be disposed adjacent to a portion 130 of the mounting portion 100, thereby sharing a common perpendicular line 138 with the fixed end 114. The perpendicular line 138 may be defined as a line drawn from the center of the hinge 126 (see below) that intersects with a cord access path 128. The free end 116 may be spaced apart from the portion 130 of the mounting portion 110 adjacent to the free end 116 to define the cord access path 128. Alternatively, the free end 116 may touch or overlap with the portion 130 of the mounting portion. In such circumstances, the cord access path becomes available upon applying a bending force on the arm to expose the cord access path. The cord access path is in communication with a cord receiving portion 124. The cord access path 128 provides a path to allow a cord to be inserted into the cord receiving portion 124. Either or both of the free end 116 and the portion 130 of the mounting portion 130 adjacent to the free end 116 may be angled 132 relative to a longitudinal line 134 to form a notch. Preferably, both are angled to form a triangular notch. The triangular notch shape of the cord access path 128 assists in guiding a cord into the cord receiving portion 124. The angle may be greater than 0° and less than 90° relative to the longitudinal line 134. In another aspect, the angle may be greater than 20° and less than 70°. In yet another aspect, the angle may be greater than 30° and less than 60°. The longitudinal line 134 may be defined as line drawn through the center of a hinge 176 (see below) that intersects with the cord access path 128. The cord-guiding notch may be radiused or curved rather than having a uniform slope. It is within the scope of the invention that any shape that facilitates cord insertion may be implemented.

The cord management device 100 further includes a cord receiving portion 124 defined by the mounting portion 110 and the arm 112. As shown in FIG. 3, the outer perimeter of the cord receiving portion 124 is defined by the inner perimeter of the arm 112 and a portion of the outer perimeter of the mounting portion 110. For example, one side of the mounting portion 110 and the entire inner perimeter of the arm 112 from fixed end 114 to free end 116, may define the cord receiving portion 124. The cord receiving portion 124 is sized to receive one or more cords 300 (FIG. 5).

The cord management device 100 may further include a hinge 126 that couples the arm 112 with the mounting portion 110. In an aspect of the present invention, the hinge 126 may comprise a living hinge. The hinge 126 provides relative bending motion between the arm 112 and the mounting portion 110. Because the arm 112 is coupled to the mounting portion 110 on the fixed end 114 by the hinge 126 and is free from the mounting portion 110 on the free end 116, the arm 112 is able to move about the hinge 126. The hinge may be configured to allow motion of the arm about the hinge in a up/down manner or a side to side manner.

FIG. 4 shows a bottom perspective view of the cord management device 100. The cord management device 100 may include one or more dimples 140 disposed on a surface of the arm 112. More particularly, the dimples 140 may be disposed on an underside of the curved portion 118 of the arm 112. The dimples 140 are sized and shaped to correspond to the size and contour of a cord. When a cord is inserted into the cord receiving portion 124, the cord may rest within one of the dimples 140. When a plurality of dimples is provided, the dimples may have different sizes from each other to accommodate different sizes of cords. The dimples may be oriented parallel to the axes of the cords so that the cords easily align with the dimples. Dimples may also be disposed on a surface of the mounting portion 110 to assist in retaining the cord in a desired position. Furthermore, Protrusions may be disposed on a top surface of curved portion 118 of the arm 112.

In another aspect the present invention, the cord management device 100 may include a second arm 162, a second hinge 176, a second cord receiving portion 174, and a second cord access path 178. The second arm 162 includes a curved portion 160, a free end 166, and a fixed end 164. Each of these features is analogous to the corresponding feature discussed above, and arranged in a similar manner. That is, the fixed end 164 of the second arm 162 may similarly be coupled to the mounting portion 110 via the hinge 176, while the free end 166 of the second arm 162 may similarly be disposed as with the free end 116 of the arm 112. The arm 112 and the second arm 162 may be coupled to opposing sides of the mounting portion so that a single cord may be retained by both arms in a single direction. The second cord receiving portion 174 may similarly be defined by the inner perimeter of the second arm 162 and a portion of the outer perimeter of the mounting portion 110. Either or both of the free end 166 of the second arm 162 and the second portion 180 of the mounting portion 110 adjacent to the free end 166 may similarly be angled 182 relative to a second longitudinal line 184 to form a notch. The second longitudinal line 184 may be defined by a line drawn from the center the hinge 126 that intersects with the second cord access path 178. The angle 182 may serve the same purpose as the discussed above with respect to the arm 112. The second arm 162 may similarly have a shape such that the free end 166 of the second arm 162 shares a second perpendicular line 188 with the fixed end 164 of the second arm 162. The second perpendicular line 188 may be defined by a line drawn from a center of the second hinge 176 that intersect with the second cord access path 178. A surface of the second arm 162 may similarly comprise one more dimples 190.

A shown in FIG. 3, the first perpendicular line 138 and the second perpendicular line 188 may be parallel to, and separated from, each other. The first longitudinal line 134 and the second longitudinal line 184 may be parallel to, and separated from, each other. Furthermore, the longitudinal line 134 may intersect both the free end 116 of the arm 112 and the fixed end 164 of the second arm 162. The second longitudinal line 184 may intersect both the fixed end 114 of the arm 112 and the free end 166 of the second arm 162.

The cord management device 100 may be formed by injection molding of substantially rigid material, such as a thermoplastic, and the like. By manufacturing the cord management device 100 by injection molding and with a substantially rigid material, the arm may be flexible enough to bend relative to the mounting portion when a bending force is applied, but rigid enough to return to the original injection molded shape once the bending force is removed. Alternatively, the cord management device may be manufactured with several other material types such as elastomeric, thermoset, and the like, as well as different manufacturing processes such as compression molded, extruded, and the like. Any material or manufacture method is suitable so long as the cord management device comprises sufficient flexibility and shape memory. In addition, the surface of the cord management device may be treated or textured in such a manner that friction or lubricity is enhanced to allow the cords to be more easily retained and/or moved within/along the surface. For example, texturing may be imbedded in the mold, while the surface may be plasma treated or treated with a lubricant.

In operation, starting from a configuration where the cord management device 100 does not have a cord 300 inserted therein, the operator holds one or more cords. Optionally with the same hand, the operator may then grip the arm 112 and bend the arm 112 about the hinge 126. Bending the arm 112 in this manner exposes or expands the size of the cord access path 128. The amount of bending necessary will depend on this size of the cord 300 being inserted. For a larger cord, more bending will be required as compared to the amount of bending required for a smaller cord because a larger expansion of the cord access path 128 will be necessary. After the operator has sufficiently expanded the cord access path 128 to fit the cord, with the same hand, the operator can then insert the cord 300 into cord receiving portion 124 via the expanded cord access path 128 such that the first arm 118 rests on top of the cord. Thus, an operator can operate insert or remove cords from the cord management device with a single hand.

Alternatively, instead of the bending the arm 112 by gripping the arm and actively bending it, the operator may simply push the cord 300 into the cord access path 128. The preferred triangular notch shape of the cord access path 128 directs the cord 300 toward the cord receiving portion 124. The continued application of force as the cord 300 is pushed further into the cord access path 128 causes the cord access path 128 to expand outwardly around the cord. Thus, the expansion of the cord access path 128 may occur with a single insertion motion, without a separate gripping of the arm 112 by the operator. Thus, an operator can operate insert or remove cords from the cord management device with a single hand.

Once the cord 300 is disposed within the cord receiving portion 124, the operator may release the bending force on the arm 112. This release may occur automatically once the cord 300 has entered the cord receiving portion 124 because the cord 300 is no longer providing an outward force from within the cord access path 128. Alternatively, the release may occur by the operator releasing the arm 112 if the operator has provided the bending force by griping the arm 112. Preferably, the tension built into the material will allow the first arm 112 to return to a rest position, which decreases the size of the cord access path 128. With the cord access path 128 size reduced, the cord 300 is unlikely to inadvertently slide out of the cord management device 100 because the cord 300 will not fit through the cord access path 128. Additionally, the presence of the dimples 140 assists in retaining the cord 300 in a set position, preferably underneath the arm 112. The cord 300 being retained within the cord management device 100 is shown in FIG. 5.

After the cord 300 has been secured within the cord receiving portion 124, the above steps may be repeated for the second arm 162 and second cord receiving portion 174. While still retaining the cord 300 in the operator's hand, the operator preferably may use the same hand pass the cord 300 over the top surface 120 of the mounting portion 110. Then, the operator may then similarly bend the second arm 162 about the second hinge 176 to expose or expand the second cord access path 178. Next, the operator may similarly insert the cord 300 into the second cord receiving portion 174 via the expanded second cord access path 178, preferably such that the second arm 163 rests on top of the cord 300. When the cord 300 is retained in both the cord retaining portions 124, 174, as shown in FIG. 5, the cord is snugly held in place.

It should be understood that the same steps may above may repeated starting with the second arm 162 instead of the arm 112. Furthermore, the above steps may be performed in reverse to remove the one or more cords from the cord management device 100.

An advantage over prior art cord management devices is that in the inventive cord management device, a single cord can be added or removed independent from other cords that may already be retained in the device.

A further advantage over prior art cord management devices is that, according to one aspect thereto, the inventive cord management device is of a generally planar configuration, free of any major protrusions extending from the generally-planar surfaces thereof. For example, referring back to FIG. 3, the cord management device 100 lies substantially in a plane that is coplanar with either top surface 120 or bottom surface 122 (FIG. 4). Although the cord management device 100 of the present invention has a thickness, it will be readily apparent to those of ordinary skill in the art, upon reading the within specification, that cord management device 100 is free of any projection, protuberance or other protrusion that extends even a nominal distance in any direction away from either top surface 120 or bottom surface 122 (FIG. 4).

The lack of any substantial protrusions extending from a generally-planar surface of the cord management device 100 provides numerous advantages. First, configuring the cord management device 100 to have a generally planar configuration provides a generally flat or smooth surface with which that cords, tubes, wires or other products used in an operating room during a surgery will not snag or become otherwise entangled therewith. This also enhances the ease with which a user may insert cords, etc., into the cord receiving portion 124 during use, and especially, during one-handed use, without becoming snagged on any such protrusion.

Moreover, configuring the cord management device 100 to have a generally planar configuration eliminates any sharp points or edges that might otherwise inadvertently tear the surgical drape 114 either while the drape 114 is being positioned over a patient or during the surgical procedure itself. This not only increases patient safety, but also reduces waste and improves operating room efficiency.

Configuring the cord management device 100 to have a generally planar configuration also prevents damage to the drape 114 while the drape is being manufactured, stored, shipped, unpacked and used. For example, most surgical drapes 114 are folded into a very specific folded-up configuration after they are manufactured, but prior to being packaged. Such a folded-up configuration provides for efficient sterilization, shipping and storage. According to most folding patterns used in the industry, a typical surgical drape 114 of the type suitable for use with the cord management device 110 of the present invention would be folded over upon itself numerous times, thereby layers of face-to-face contact with the surgical drape 114 on itself. A cord management device 110, such as the one described herein, typically is affixed to the surgical drape 114 prior to the surgical drape 114 being folded as described herein. As such, a cord management device 110, such as the one described herein, would be embedded within the multiple layers of the surgical drape 114 after it has been folded upon itself numerous times. Any protrusions extending from any surface of the cord management device 110 would likely rub against the material that has been folded over it or may otherwise penetrate or tear the surgical drape 114 material. Accordingly, configuring the cord management device 110 such that it is in a generally planar configuration reduces the risk that such a device, when embedded within a folded-up surgical drape 114, will tear or otherwise damage the drape 114.

The cord management device may be radiopaque so that if a piece breaks off it can easily be found using x-rays. The entire device or any portion of the device may be radiopaque.

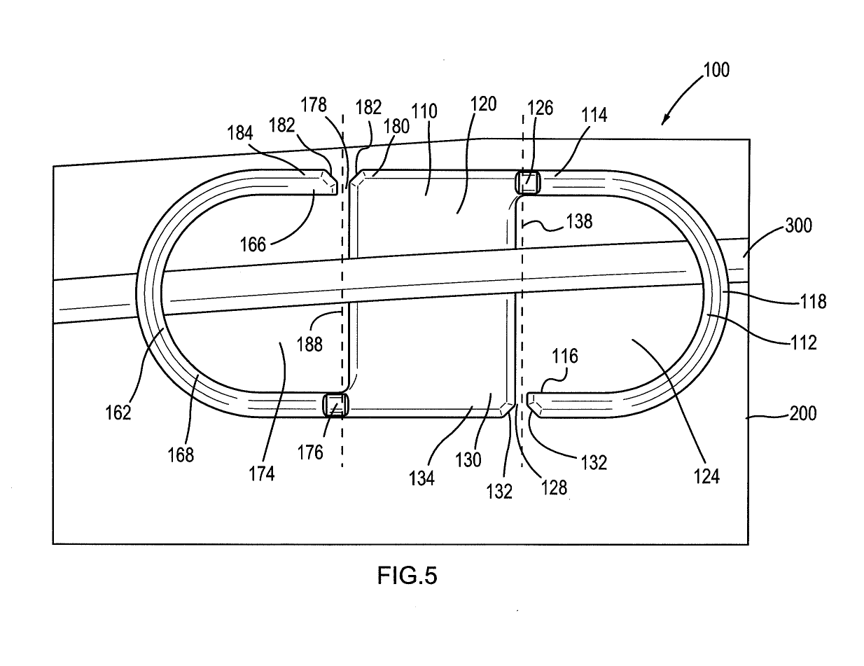

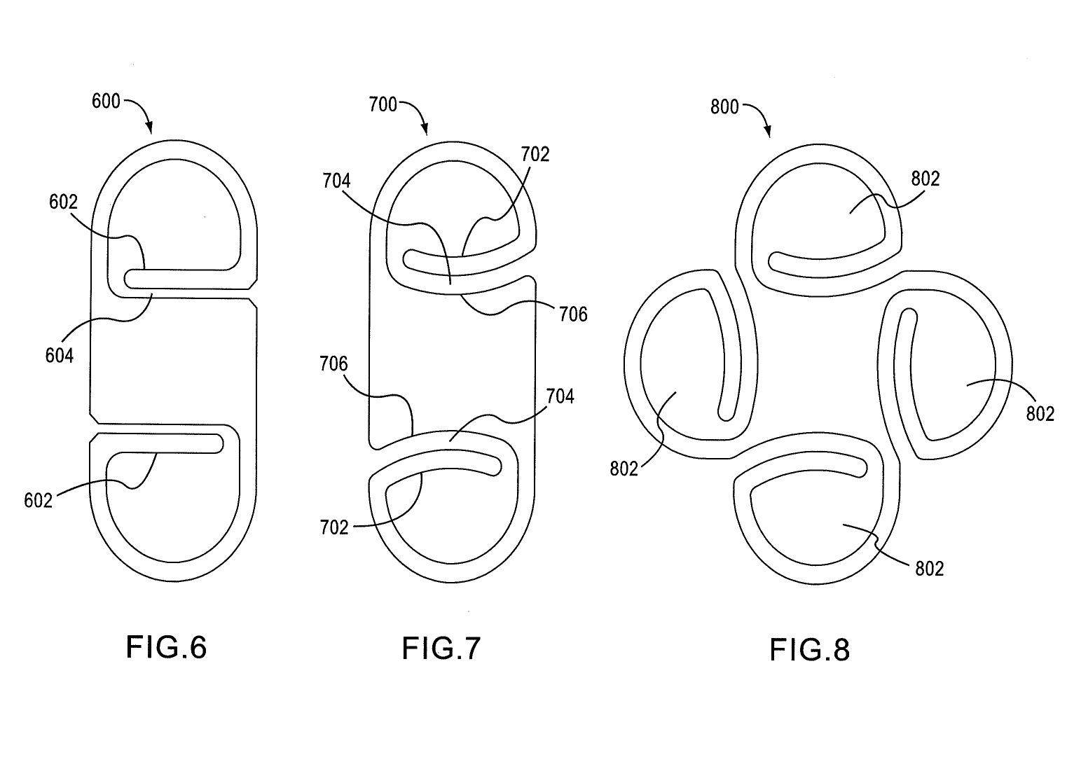

FIG. 6 shows a cord management device 600 in accordance with another aspect of the present invention. The cord management device 600 is similar to the cord management device 100 shown in FIGS. 3-5. It is readily apparent from FIG. 6 that many of the features discussed above may also be present in the cord management device 600. For example the cord management device 600 may include first and second arms having optional dimples on the surfaces, a mounting portion having optional dimples on a surface, notches, cord receiving portions, cord access paths, and hinges, among other features. The description of these features may be found above, and are omitted for brevity. The cord management device 600 may further include one or more securing portions 602. As shown in FIG. 6, the arm may include a securing portion extending from the free end of the arm and optionally terminating adjacent to the fixed end of the arm. The securing portion 602 may extend substantially parallel to an edge of the mounting portion, thereby providing a cord access path 604 that extends along the length of the mounting portion. The securing portion 602 and extended cord access path 604 provides additional protection against inadvertent removal of a cord retained within the cord retaining portion. The cord management device is operated substantially the same as described above, except that the cord must travel within the cord access path 604 along the length of the securing arm 602, before entering the cord retaining portion. Once present in the cord retaining portion, the cord must again travel within the cord access path 604 before exiting the cord management device.

FIG. 7 shows a cord management device 700 in accordance with another aspect of the present invention. The cord management device 700 is similar to the cord management device 600 shown in FIG. 6. It is readily apparent from FIG. 7 that many of the features discussed above may also be present in the cord management device 700. For example the cord management device 700 may include first and second arms having optional dimples on the surfaces, a mounting portion having optional dimples on, a surface, notches, cord receiving portions, cord access paths, hinges, and securing portions, among other features. The description of these features may be found above, and are omitted for brevity. The cord management device 700 includes the feature that the securing portions 702 may be curved. As shown in FIG. 7, both the securing portion 702 and the edge 706 of the mounting portion may be curved. The curvature of the securing portion 702 and the edge 706 may be the same, thereby providing a curved cord access path 704. The curvature of the curved cord access path 704 more closely mirrors the shape of a cord and may allow for smoother insertion.

FIG. 8 shows a cord management device 800 in accordance with another aspect of the present invention. The cord management device 800 is similar to the cord management device 700 shown in FIG. 7. It is readily apparent from FIG. 8 that many of the features discussed above may also be present in the cord management device 800. For example the cord management device 800 may include first and second arms having optional dimples on the surfaces, a mounting portion having optional dimples on a surface, notches, cord receiving portions, cord access paths, hinges, and securing portions, among other features. The description of these features may be found above, and are omitted for brevity. The cord management device 800 includes the feature that there are four arms and four cord retaining portions 802. As shown in FIG. 8, a single mounting portion may have four sides, and each side may have an extending arm that, along with the mounting portion, defines a cord retaining portion 802. The arms form the cord retaining portions 802 in the same manner as described above. With this structure, the cord management device 800 can retain cords in two directions substantially perpendicular to each other. A first cord could pass through two of the cord retaining portions that oppose each other in a first direction, and a second cord could pass through the remaining two cord retaining portions that oppose each other in a second direction. In an aspect the first direction may be a horizontal direction and the second direction may be a vertical direction. In another aspect, cords could pass through adjacent cord retaining portions.

FIGS. 9a, 9b show a cord management device 900 in accordance with another aspect of the present invention. The cord management device 900 is similar to the cord management device 100 shown in FIGS. 3-5. It is readily apparent from FIGS. 9a, 9b that many of the features discussed above may also be present in the cord management device 900. For example the cord management device 900 may include first and second arms having optional dimples on the surfaces, a mounting portion having optional dimples on a surface, cord receiving portions, and hinges, among other features. The description of these features may be found above, and are omitted here for brevity. The cord management device 900 includes a locking mechanism 902. As shown in FIG. 9b, the locking mechanism 902 may include a projection 904 extending from the free end of the arm and a corresponding projection receiving portion 906 formed within the mounting portion. The projection 904 is shaped to mate with the corresponding projection receiving portion 906. Alternatively, the mounting portion may have a projection and the arm may have the projection receiving portion. In addition, other locking mechanisms known in the art could be used.

As shown in FIG. 9a, in order to mate with the mounting portion, instead of terminating adjacent to the mounting portion, the free end of the arm may extend along the length of the mounting portion so that when the projection 904 is mated with the projection receiving portion 906, a portion of the arm forms a continuous edge with the mounting portion. As shown in FIG. 9b, the locking mechanism 904 is unlocked by pivoting the arm such that the projection 904 is removed from the projection receiving portion 906, thereby providing a path 908 for the cord to be inserted. As shown in FIG. 9b, the projection 904 may be generally rounded and slightly wider than an entryway of the receiving portion 906. Due the above-described flexibility of the material, when force is applied on the free end of the arm to insert the projection 904 in to the projection receiving portion 906, the entryway of the projection receiving portion is expanded slightly to allow the projection 904 to enter. Once the projection 904 if fully inserted into the projection receiving portion 906, the force is removed, and the projection receiving portion will return to the original shape, thereby securely locking the arm in place.

FIGS. 10a, 10b show a cord management device 1000 in accordance with another aspect of the present invention. The cord management device 1000 is similar to the cord management device 900 shown in FIGS. 9a, 9b. It is readily apparent from FIGS. 10a, 10b that many of the features discussed above may also be present in the cord management device 1000. For example the cord management device 1000 may include first and second arms having optional dimples on the surfaces, a mounting portion having optional dimples on a surface, cord receiving portions, and hinges, among other features. The description of these features may be found above, and are omitted for brevity. The cord management device 1000 includes an overlapping arm to limit cord travel out of the device. Optionally it may include the feature that the locking mechanism 1002 allows the arm to mate with mounting portion along a surface of the mounting portion. As shown in FIG. 10b, the locking mechanism 1002 may include an arm receiving portion 1004 formed within a surface of the mounting portion that is sized to receive a portion of the arm. The arm receiving portion 1004 may further include a projection receiving portion 1006 sized to receive a projection 1008 extending from a surface of the arm.

As shown in FIG. 10a, in order to mate with the mounting portion, instead of terminating adjacent to the mounting portion, the free end of the arm may extend along the length of the mounting portion so that when the free end of the arm is mated with the arm receiving portion 1004, a portion of the arm forms a continuous edge with the mounting portion. As shown in FIG. 10b, the locking mechanism 1002 may be unlocked by pivoting the arm such that the free end of the arm is removed from the arm receiving portion 1004, thereby providing a path 1010 for the cord to be inserted. To lock the locking mechanism 1002, the arm is pivoted so that the free end of the arm mates with the arm receiving portion 1004. Additionally, once the arm is positioned into the arm receiving portion 1004, the projection 1008 extending from a surface of the arm may mate with the projection receiving portion 1006, thereby providing a secure connection.

FIG. 11 shows a cord management device 1100 in accordance with another aspect of the present invention. The cord management device 1100 is similar to the cord management device 100 shown in FIGS. 3-5. It is readily apparent from FIG. 11 that many of the features discussed above may also be present in the cord management device 1100. For example the cord management device 1100 may include first and second arms having optional dimples on the surfaces, a mounting portion having optional dimples on a surface, notches, cord receiving portions, cord access paths, and hinges, among other features. The description of these features may be found above, and are omitted for brevity. The cord management device 1100 includes the feature that the fixed end of the first arm sharing a common longitudinal line with free end of the second arm, the fixed end of the first arm shares a common longitudinal line 1102 with the fixed end of the second arm. As shown in FIG. 11, the cord management device is substantially symmetrical relative to a horizontal axis 1104.

It should be understood that many of the features discussed above with respect to the various aspects of the present invention may be incorporated into other aspects. Any feature that is not mutually exclusive relative to another feature may be included in the same device. For example the four-arm arrangement of FIG. 8 could be applied to the cord management devices shown in FIGS. 3-6 and 9a-11. Similarly, the overlapping arms and/or locking feature of FIGS. 10a, 10b may be applied to the cord management devices shown in FIGS. 6-8 and 11. The fixed end of the arms being disposed along the same longitudinal line feature of FIG. 11 can be applied to the cord management devices of FIGS. 3-10b.

Example aspects have been described in accordance with the above advantages. It will be appreciated that these examples are merely illustrative of aspects of the invention. Many variations and modifications will be apparent to those skilled in the art.

1. A cord management device adapted for use with a surgical drape, comprising:
a mounting portion adapted to be affixed to a surface of the surgical drape;
a substantially rigid arm having a fixed end and a free end, wherein the arm is coupled to the mounting portion at the fixed end, and the free end is free from the mounting portion; and
wherein the mounting portion and the arm define a cord receiving portion.

2. The cord management device of claim 1, wherein the fixed end of the arm is coupled to the mounting portion via a hinge.

3. The cord management device of claim 2, wherein the free end of the arm and a portion of the mounting portion adjacent to the free end of the arm define a cord access path.

4. The cord management device of claim 3, wherein at least one of the free end of the arm and the portion of the mounting portion adjacent to the free end of the arm is shaped to form a notch.

5. The cord management device of claim 3, wherein the hinge permits pivotal movement of arm about a perpendicular line and wherein the cord access path lies along the perpendicular line.

6. The cord management device of claim 1, further comprising at least one dimple disposed on a surface of the arm and sized to receive a cord.

7. The cord management device of claim 2,
wherein the hinge comprises a living hinge.

8. The cord management device of claim 1,
wherein the arm comprises an arcuate shape.

9. The cord management device of claim 1,
wherein said arm is a first arm;
and wherein said cord management device further comprises:
a second arm having a fixed end and a free end, wherein the second arm is coupled to the mounting portion at the fixed end of the second arm, and the free end of the second arm is free from contacting the mounting portion; and
wherein the mounting portion and the second arm define a second cord receiving portion.

10. The cord management device of claim 9,
wherein the free end of the first arm and a first portion of the mounting portion adjacent to the free end of the first arm define a first cord access path, and
wherein the free end of the second arm and a second portion of the mounting portion adjacent to the free end of the second arm define a second cord access path.

11. The cord management device of claim 10, wherein a line extending from the free end of the first arm to the free end of the second arm intersects a line drawn between the first and second portions of the mounting portion.

12. The cord management device of claim 11, wherein a line extending from the free end of the first arm to the free end of the second arm is parallel to a line drawn between the first and second portions of the mounting portion.

13. The cord management device of claim 1, wherein the arm is made from a pre-tensioned thermoflexible plastic.

14. The cord management device of claim 1, wherein the mounting portion is substantially rigid.

15. The cord management device of claim 1, wherein a horizontal plane intersects a surface of the mounting portion and a surface of the arm.

16. The cord management device of claim 1, wherein the arm includes a securing portion extending toward the fixed end of the arm.

17. The cord management device of claim 1, wherein the cord management device further includes a locking mechanism configured to secure the arm to the mounting portion.

18. The cord management device of claim 17, wherein the locking mechanism includes a receiving portion disposed on a surface of the mounting portion.

19. The cord management device of claim 16, wherein the securing portion extends adjacent an edge of the mounting portion, and wherein the securing portion and the edge of the mounting portion are curved.

20. The cord management device of claim 9, further comprising:
a third arm having a fixed end and a free end, wherein the third arm is coupled to the mounting portion at the fixed end of the third arm, and the free end of the third arm is free from the mounting portion;
a fourth arm having a fixed end and a free end, wherein the fourth arm is coupled to the mounting portion at the fixed end of the fourth arm, and the free end of the fourth arm is free from the mounting portion;
wherein the mounting portion and the third arm define a third cord receiving portion; and
wherein the mounting portion and the fourth arm define a fourth cord receiving portion.

21. The cord management device of claim 1,
wherein the arm and the second arm are coupled to opposing sides of the mounting portion, and
wherein the third arm and the fourth arm are coupled to opposing sides of the mounting portion.

22. A method of securing a cord to a surgical drape, the method comprising:
providing a cord management device affixed the surgical drape, the cord management device comprising:
a mounting portion contacting a surface of the surgical drape;
an arm having a fixed end and a free end, wherein the arm is coupled to the mounting portion at the fixed end, and the free end is free from the mounting portion; and
wherein the mounting portion and the arm define a cord receiving portion;
expanding the cord access path by applying a bending force on the arm;
passing a cord through the cord access path;
placing the cord in the cord receiving portion after passing the cord through the cord access path; and
releasing the bending force on the arm, thereby retaining the cord within the cord receiving portion.

23. The method of claim 22, wherein an outward force created by forcing the cord through the cord access path provides the bending force on the arm.

24. The method of claim 22, wherein the bending force is applied by gripping the arm before passing the cord through the cord access path.

25. The method of claim 22, further comprising placing the cord within a dimple formed on a surface of the arm.

26. The method of claim 22, wherein the cord access path comprises at least one notch.

27. The method of claim 22, wherein the bending force is applied by bending the arm about a hinge.

28. The method of claim 22,
wherein the cord management device further comprises:
a second arm having a fixed end and a free end, wherein the second arm is coupled to the mounting portion at the fixed end of the second arm, and the free end of the second arm is free from the mounting portion; and
wherein the mounting portion and the second arm define a second cord receiving portion,
the method further comprising:
expanding the second cord access path by applying a bending force on the second arm;
passing the cord through the second cord access path;
placing the cord in the second cord receiving portion after passing the cord through the second cord access path; and
releasing the bending force on the second arm, thereby retaining the cord within the second cord receiving portion.

29. The method of claim 22, wherein the arm of the cord management device is made from a substantially rigid material.

30. The method of claim 22, wherein the cord management device is mounted to the surgical drape by the manufacturer.

31. The method of claim 22, wherein at least one of the steps is performed using one hand.

32. The method of claim 22, wherein the cord comprises a first cord and wherein the method further comprises the steps of:
expanding the cord access path by applying a bending force on the arm;
passing a second cord through the cord access path;
placing the second cord in the cord receiving portion after passing the second cord through the cord access path; and
releasing the bending force on the arm, thereby retaining the second cord within the cord receiving portion;
wherein the first cord remains secured in the cord receiving portion while the second cord is added.

33. The method of claim 22, wherein the arm includes a securing portion extending toward the fixed end of the arm.

34. The method of claim 22, further comprising locking the arm to the mounting portion.

35. The method of claim 34, wherein locking the arm comprises placing the arm into a receiving portion disposed on a surface of the mounting portion.

36. The method of claim 33, wherein the securing portion extends adjacent an edge of the mounting portion, and wherein the securing portion and the edge of the mounting portion are curved.

37. The method of claim 28,
wherein the cord comprises a first cord;
wherein the cord management device further comprises:
a third arm having a fixed end and a free end, wherein the third arm is coupled to the mounting portion at the fixed end of the third arm, and the free end of the third arm is free from the mounting portion;
a fourth arm having a fixed end and a free end, wherein the fourth arm is coupled to the mounting portion at the fixed end of the fourth arm, and the free end of the fourth arm is free from the mounting portion;
wherein the mounting portion and the third arm define a third cord receiving portion;
wherein the mounting portion and the fourth arm define a fourth cord receiving portion
the method further comprising:
expanding the third cord access path by applying a bending force on the third arm;
passing a second cord through the third cord access path;
placing the second cord in the third cord receiving portion after passing the second cord through the third cord access path;
releasing the bending force on the third arm, thereby retaining the second cord within the third cord receiving portion;
expanding the fourth cord access path by applying a bending force on the fourth arm;
passing the second cord through the fourth cord access path;
placing the fourth cord in the fourth cord receiving portion after passing the second cord through the fourth cord access path; and
releasing the bending force on the fourth arm, thereby retaining the second cord within the fourth cord receiving portion.
